(19) United States
(12) Patent Application Publication
(10) Pub. No.: US 2011/0125210 A1
(43) Pub. Date: May 26, 2011

(54) LTCC/HTCC HYBRID FEEDTHROUGH

(75) Inventors: Richard W. A. Francis, White Bear Lake, MN (US); Rajesh V. Iyer, Eden Prairie, MN (US)

(73) Assignee: Medtronic, Inc., Minneapolis, MN (US)

(21) Appl. No.: 12/625,271

(22) Filed: Nov. 24, 2009

(51) Int. Cl. A61N 1/375; B32B 9/04; C03B 29/00

(52) U.S. Cl. 607/37; 428/188; 156/89.11

(57) ABSTRACT

The present invention is directed to a ceramic monolith for use as a feedthrough in medical devices and method of making. The monolith includes a first surface, a second surface, and a passageway extending from the first surface to the second surface. The first surface is high temperature co-fired ceramic and the second surface is low temperature co-fired ceramic, and the two ceramics are intermixed in a blended interface located between the first and second surfaces.

### FIELD OF THE INVENTION

The present invention generally relates to ceramic feedthroughs, and specifically, to a simplified, improved composite ceramic feedthrough including low and high temperature ceramics adapted to provide an isolated interaction between devices and a remote site via a passageway for a variety of implantable therapy delivery devices, diagnostic devices, and other devices.

The invention particularly relates, in a specific embodiment, to ceramic feedthroughs used in electronic applications in implantable medical devices, and methods of making ceramic feedthroughs.

### BACKGROUND OF INVENTION

Ceramic feedthroughs have been used extensively in a number of devices, notably implantable medical devices such as pacemakers, defibrillators, and neurostimulators. Common ceramic feedthrough designs include an array of electrical conductors insulated from one another by a dielectric; the dielectric attached to a conductive ferrule, the ferrule facilitating hermetic attachment to external packaging for the implantable therapy delivery and diagnostic devices and providing a ground/earth connection through patient body tissue.

Both low temperature co-fire ceramic (LTCC) and high temperature co-fire ceramic (HTCC) technologies are utilized in many current feedthroughs. LTCC is a ceramic generally sintered below 1000 Celsius, while HTCC is a ceramic sintered at around 1600 Celsius. In LTCC, a quantity of glass is sometimes added to a ceramic powder to facilitate temperature liquid-phase sintering. Actual temperatures may vary. Feedthroughs combining LTCC and HTCC typically have connected discrete layers of LTCC and HTCC using a variety of techniques, including glue, epoxy, and lamination. Feedthroughs combining LTCC and HTCC in discrete layers have the drawbacks of expensive and time-consuming production, delamination or other disconnection, a non-unitary feedthrough, large size, and necessarily wide spacing between LTCC-HTCC electrical connections.

### SUMMARY OF INVENTION

In one aspect, the present invention is directed to a ceramic monolith which includes a first surface including a high temperature co-fired ceramic (HTCC), a second surface including a low temperature co-fired ceramic (LTCC) and a passageway extending from the first surface to the second surface. The monolith also includes a blended interface located between the first and second surfaces, which includes intermixed HTCC and LTCC. The blended interface contains more LTCC disposed proximate to the second surface than proximate to the first and more HTCC disposed proximate to the first surface than proximate to the second surface.

In one embodiment, the LTCC and HTCC are interspersed within the monolith such that the ratio of LTCC to HTCC increases with distance from the first surface toward the second surface.

In one embodiment, the first and second surfaces are substantially parallel to one another. In another embodiment the first and second surfaces are substantially perpendicular to one another.

In one embodiment, the passageway extends entirely out of the first and second surfaces. In another embodiment, the passageway does not extend out of the first surface.

In one embodiment, the ceramic monolith further includes a ceramic or metal augmenting member within the monolith.

In one embodiment, the ceramic monolith is part of an implantable medical device.

In one embodiment, the passageway is an electrical feedthrough. In another embodiment, the passageway is a chemical feedthrough.

In another aspect, the present invention is directed to a ceramic monolith which includes a surface including a high temperature co-fired ceramic (HTCC) portion, a low temperature co-fired ceramic (LTCC) portion and a passageway extending from the HTC portion to the LTCC of the surface. The monolith also includes a blended interface which includes intermixed HTCC and LTCC. In one embodiment, the monolith is part of an implantable medical device.

In another aspect, the invention is directed to implantable medical device which includes a feedthrough. The feedthrough includes a first surface including a high temperature co-fired ceramic (HTCC), a second surface including a low temperature co-fired ceramic (LTCC), and a blended interface located between the first and second surfaces. The blended interface includes intermixed HTCC and LTCC such that there is more LTCC disposed proximate to the second surface than proximate to the first surface and more HTCC disposed proximate to the first surface than proximate to the second surface.

In one embodiment, the feedthrough is an electrical feedthrough and includes an electrical connection extending between the first surface and the second surface. In one embodiment, the electrical feedthrough includes one or more electrical components within the electrical feedthrough and in communication with the electrical connection. In another embodiment, the electrical feedthrough includes at least one capacitor connected to an electrical ground as a part of the electrical connection. In another embodiment, the electrical connection is adapted to serve as a conduit for transmission of electrical energy. In another embodiment, the feedthrough includes a pattern of electrical connections in a plane between the first and second surfaces.

In another embodiment, the feedthrough is a chemical feedthrough and comprises a passageway extending between the first surface and the second surface. In one embodiment, the passageway is an aperture extending from the first surface to the second surface. In another embodiment, the feedthrough includes a pump located in the passageway.

In another aspect, the present invention is directed to a method of making a ceramic monolith. The method includes
a) firing a high temperature co-fired ceramic (HTCC) material to form a HTCC portion having a first surface and a second surface, the second surface having a porous region;
b) forming a passageway that extends from the first surface to the second surface of the HTCC portion;
c) providing an unfired low temperature co-fired ceramic (LTCC) material where the unfired LTCC has a sintering temperature, a first surface, and a second surface;
d) forming a passageway that extends from the first surface of the unfired LTCC material to the second surface of the unfired LTCC material;
e) mating the unfired LTCC and the fired HTCC portion together such that the passageway region on the second surface of the HTCC portion aligns with the passageway region on the second surface of the unfired LTCC to create a resulting unit with a passageway from the first surface of the HTCC portion to the first surface of the unfired LTCC; and
f) firing the resulting unit at a temperature higher than the sintering temperature of the unfired LTCC such that the LTCC material infiltrates the second surface of the HTCC portion and creates an LTCC/HTCC portion, and the unfired LTCC sinters into a LTCC portion.

In one embodiment, method includes introducing a conductor between the unfired LTCC material and the HTCC portion to aid formation of an electrically conductive connection between the first surface of the HTCC portion and the first surface of the unfired LTCC material. In one embodiment, the method further includes mating the unfired LTCC and the HTCC portion together such that the electrically conductive region on the second surface of the HTCC portion aligns with the electrically conductive region on the second surface of the unfired LTCC to create a resulting unit with an electrically conductive connection from the first surface of the HTCC portion to the first surface of the unfired LTCC.

In one embodiment, the method includes mating the unfired LTCC and the HTCC portion together such that the passageway on the second surface of the HTCC portion aligns with the passageway on the second surface of the unfired LTCC to create a resulting unit with a passageway from the first surface of the HTCC portion to the first surface of the unfired LTCC.

The porous region of the second surface of the HTCC portion can be created in many ways. In one embodiment, the second surface of the HTCC portion is made porous by compressing the unfired HTCC material to create a density gradient before firing. In another embodiment, the second surface of the HTCC portion is made porous by using different sized granules throughout the HTCC before firing. In another embodiment, the second surface of the HTCC portion is made porous by including organic compounds in the HTCC material to burn out during firing. In another embodiment, the second surface of the HTCC portion is made porous by including unfired LTCC material in the HTCC material to burn out during firing. In another embodiment, the second surface of the HTCC portion is made porous by abrading the second surface of the HTCC portion after firing. In another embodiment, the second surface of the HTCC portion is made porous by including inorganic material in the unfired HTCC to burn out during firing. In another embodiment, the second surface of the HTCC portion is made porous by plasma treating the second surface of the HTCC portion after firing. In another embodiment, the second surface of the HTCC portion is made porous by chemical etching the second surface of the HTCC portion after firing. In another embodiment, the second surface of the HTCC portion is made porous by optically ablating the second surface of the HTCC portion after firing. In another embodiment, the second surface of the HTCC portion is made porous by heat ablating the second surface of the HTCC portion after firing.

Implantable medical devices of the invention include, but are not limited to, pacemakers, implantable cardioverter-defibrillators, implantable neurostimulators, implantable electrical stimulation devices, implantable pulse generators, and drug pumps for long-term sustained drug delivery, such as the SynchroMed® implantable infusion system (Medtronic, Inc., Minneapolis, Minn.), the Concerto® Cardiac Resynchronization Therapy Defibrillator (Medtronic, Inc., Minneapolis, Minn.), and the Activa® PC Neurostimulator (Medtronic, Inc., Minneapolis, Minn.). An implantable device of the invention has a body tissue or fluid-contacting surface.

Other features, objects and advantages of the present invention will be in part apparent to those skilled in art and in part pointed out hereinafter. All references cited in the instant specification are incorporated by reference for all purposes. Moreover, as the patent and non-patent literature relating to the subject matter disclosed and/or claimed herein is substantial, many relevant references are available to a skilled artisan that will provide further instruction with respect to such subject matter.

### BRIEF DESCRIPTION OF THE DRAWINGS

FIG. 1 is a cross-section of one embodiment of the present invention.

FIG. 2 is a cross-section of one embodiment of the present invention.

FIG. 3 is a cross-section of one embodiment of the present invention including a low-pass filter.

FIG. 4 is a cross-section of one embodiment of the present invention.

FIG. 5 is a cross-section of one embodiment of the present invention.

FIGS. 6a and 6b are cross-sections of two embodiments of the present invention. The 6a embodiment includes a passageway that does not extend completely from second to first surface; while the 6b embodiment includes a metal-lined passageway, sealed at one end, extending from the second surface to beyond the first surface.

FIG. 7 is a flow chart illustrating one embodiment of a method of the present invention.

FIGS. 8a, 8b, and 8c illustrate in cross-section three embodiments of electrical interconnects for HTCC portions.

FIG. 9 is a cross-section of one embodiment of the present invention illustrating a feedthrough for an implantable medical device intended for chemical delivery.

FIG. 10 is an implanted cardiac pacemaker embodying the present invention.

FIG. 11 is a cross-section of an embodiment of the present invention in which the LTCC-HTCC interface is non-planar.

FIG. 12 is a cross-section of an embodiment of the present invention including a non-planar region of HTCC-LTCC intermixture.

FIG. 13 is a cross-section of one embodiment of the invention, an implantable medical device with hybrid ceramic feedthrough and low-pass filter.

FIG. 14 is a visual flow chart illustrating steps in construction of one embodiment of the invention.

FIG. 15 is a cross-section of one embodiment of a prior-art ceramic feedthrough.

In all figures in cross-section, an unhashed portion is substantially LTCC, a partially hashed portion represents HTCC and LTCC intermixed, and a fully-hashed portion represents substantially HTCC.

Figures are not necessarily to scale.

### DETAILED DESCRIPTION OF THE INVENTION

Cardiac pacemakers and other such implantable medical devices (e.g., cochlear implants, defibrillators, neurostimulators, active drug pumps, etc.) typically comprise a hermetically sealed container and a feedthrough assembly having one or more feedthrough terminals (e.g., niobium pins) that provide conductive paths from the interior of the container (e.g., from an anode lead embedded in an internal anode) to one or more lead wires exterior to the device. In the case of a cardiac pacemaker for example, these lead wires conduct pacing pulses to cardiac tissue and/or detect cardiac rhythms. In general, such feedthrough assemblies comprise a ferrule that secures the assembly relative to the container and an insulating structure (e.g., a glass or ceramic body) that insulates the terminal pin or pins from the ferrule. The feedthrough assembly may be hermetically sealed to prevent body fluids from seeping into the device. To reduce the effects of stray electromagnetic interference (EMI) signals that may be collected by lead wires coupled to the feedthrough terminal pins, it is known to attach a discoidal capacitor to a feedthrough assembly (a discrete discoidal capacitor for a unipolar feedthrough assembly or a monolithic discoidal capacitor for a multipolar feedthrough assembly) with a ground connection. The attached capacitor serves as an EMI filter that permits passage of relatively low frequency electrical signals while shunting undesired high frequency interference signals to the ground, which may be attached to the implantable medical device container.

It is to be understood that the invention is generally applicable to any implantable medical device, additionally including, for example, medical leads, catheters, neurostimulators, deep-brain stimulators, gastic stimulation devices, implantable pulse generators, implantable cardioverter defibrillators, and pacemakers.

Implantable medical devices include but are not limited to implantable cardiac pacemakers such as those disclosed in U.S. Pat. No. 5,158,078 to Bennett et al, U.S. Pat. No. 5,312,453 to Shelton et al, or U.S. Pat. No. 5,144,949 to Olson, all hereby incorporated herein by reference in their respective entireties.

Implantable medical devices include but are not limited to PCDs (Pacemaker-Cardioverter-Defibrillators) corresponding to any of the various commercially available implantable PCDs. The present invention may be practiced in conjunction with PCDs such as those disclosed in U.S. Pat. No. 5,545,186 to Olson et al., U.S. Pat. No. 5,354,316 to Keimel, U.S. Pat. No. 5,314,430 to Bardy, U.S. Pat. No. 5,131,388 to Pless or U.S. Pat. No. 4,821,723 to Baker et al., all hereby incorporated herein by reference in their respective entireties.

Alternatively, an implantable medical device may be an implantable nerve stimulator or muscle stimulator such as that disclosed in U.S. Pat. No. 5,199,428 to Obel et al., U.S. Pat. No. 5,207,218 to Carpentier et al. or U.S. Pat. No. 5,330,507 to Schwartz, or an implantable monitoring device such as that disclosed in U.S. Pat. No. 5,331,966 issued to Bennet et al., all of which are hereby incorporated by reference herein in their respective entireties.

Ceramic feedthroughs have been used in a number of applications in the past. Prior implementations of ceramic feedthroughs in medical devices have been limited by both the necessity of biocompatibility for anything contacting blood or tissue and the utility of including heat-sensitive components into the feedthrough. LTCC usually lacks biocompatibility, and HTCC sinters at a temperature which destroys many heat-sensitive components. LTCC and HTCC often shrink or expand at different ratios during firing. Combining LTCC and HTCC into a single unit, with LTCC containing heat-sensitive components and HTCC material contacting blood or tissue, is an apparent solution to this problem.

Forming a satisfactory bond between LTCC and HTCC has traditionally been a problem. Solutions for joining LTCC and HTCC together through lamination, epoxy, or external compression results in separation of the LTCC and HTCC layers, and a large feedthrough with a complicated manufacturing process. The present invention provides a solution to this problem.

The weaknesses of LTCC in terms of biocompatibility and HTCC in terms of high sintering temperature are overcome by production of LTCC-HTCC hybrid structures possessing dual benefits of the LTCC and HTCC systems, while also reducing the number of components and processing steps required. The hybrid LTCC and HTCC monolith claimed in this invention can be produced through the following steps:

1) The unfired HTCC material structure is sintered into a ceramic monolith with electrical conducts being added at a later stage, e.g. metallic electrical connectors are brazed in position;

2) All high-temperature processing is completed at the HTCC sub-assembly stage, and may include brazing into a ferrule;

3) The mating surface of the HTCC is adapted to provide a porous interface to accept the corresponding face of the LTCC.

4) The unfired LTCC material is fabricated, incorporating any heat-sensitive components, electrical vias, and the like;

5) The unfired LTCC material and HTCC are mated together so that interconnects on the mating sides coincide; and

6) The entire unit is subjected to low temperature processing to sinter the unfired LTCC material.

The porous interface of the HTCC mating surface can be created in a variety of ways. Regardless of the preparation route adopted, the porous mating surface of the HTCC material structure permits and promotes infiltration of a liquid-sinter glass phase which bridges the interface between the LTCC and HTCC material structure during the low temperature processing step.

In one embodiment, the porous interface of the unfired HTCC material mating surface is created by compressing unfired HTCC material to create unequal pressure throughout, resulting in a porous area after firing. In another embodiment, a porous area is created by using different sized granules of unfired HTCC material at different locations. In another embodiment, the porous area is created by including organic compounds in the unfired HTCC material to burn out during the high temperature firing step. In another embodiment, the porous area is created by including unfired LTCC material granules with the unfired HTCC material, such that the LTCC burns out during the HTCC firing step. In yet another embodiment, the porous area is created by including other materials to burn out during the high temperature firing step. In another embodiment, the porous area is created by mechanically or chemically, abrading the surface after firing the HTCC.

In one embodiment, the LTCC is subjected to external force during the low temperature processing step to promote infiltration of the LTCC material into the porous region on the HTCC material. The LTCC could be subjected to weight, pressure from a spring or clamp, exposed to a high-pressure atmosphere, or acceleration to promote infiltration. In another embodiment, hot isostatic pressing is utilized to promote infiltration.

In one embodiment, the feedthrough can be assembled by firing two units of HTCC, providing a porous surface on one side of each of the HTCC material units, sandwiching these fired HTCC material units on either side of an unfired LTCC material unit with the porous sides of the HTCC units contacting the unfired LTCC material, firing the whole resulting unit at a temperature above the sintering temperature of the LTCC, and cutting the LTCC layer in half such that two LTCC-HTCC hybrid monoliths result. Alternately, one of the HTCC material layers could be removed, leaving a single LTCC-HTCC pair, or unfired LTCC material could be sandwiched around a HTCC unit, and then post-firing the HTCC layer cut or one of the LTCC layers removed.

In one embodiment, the hybrid LTCC/HTCC feedthrough of the present invention has several advantages over prior devices, including cost-savings in production, an integrated monolithic structure, reduced size, handling reliability, easily mechanized production, durability, control of stress from shrinking, closer spacing of connections in the intermediate layer, and ability to test before firing the LTCC portion.

In one embodiment, the hybrid feedthrough is between about the following in rectangular cube form: Length: 0.076 cm and 3.81 cm, Width: 0.076 cm and 0.635 cm, Height: 0.0381 cm and 0.635 cm, Surface area 0.026 square cm and 30.64 square cm. These dimensions are exemplary and not all embodiments are limited to these sizes.

In one embodiment, the hybrid feedthrough is between about the following in cylinder form: Diameter: 0.0381 cm and 0.635 cm, Height: 0.0508 cm and 3.81 cm, Surface area: 0.0084 square cm and 8.23 square cm. These dimensions are exemplary and not all embodiments are limited to these sizes.

In one embodiment, a monolith of the present invention is adapted to function as an electrical feedthrough for an implantable medical device, such as a pacemaker. A capacitor is incorporated into the feedthrough to filter any incoming signal and prevent undesired external electromagnetic signals from interfering with or corrupting the functioning of the pacemaker the feedthrough is connected to. In this embodiment, the feedthrough takes the form of a cylindrical ceramic monolith, with the planar surfaces being the first and second surfaces. While the opposing first and second surfaces are pure HTCC and LTCC respectively, HTCC and LTCC are interspersed between the two surfaces, with the area equidistant between the first and second surfaces being roughly half LTCC material and half HTCC material. The electrical connection exists between the first and second surfaces, with the capacitor included in the connection in a pure LTCC portion of the monolith proximate to the second surface. In one embodiment, the feedthrough is intended to contain substantially no substances above the limits imposed by the European Union Restriction of Hazardous Substances Directive.

While the monolith has LTCC and HTCC intermixed such that more LTCC disposed proximate to the second surface than the first surface, this does not mean that there is necessarily a linear relationship between distance and LTCC/HTCC ratio. The LTCC to HTCC ratio may vary linearly, it may have near-discrete qualities, or it may have a non-linear relationship.

In one embodiment, an implantable medical device such as a drug delivery device uses a hybrid feedthrough of the present invention as a chemical feedthrough. A pump is incorporated into the feedthrough to assist in transferring liquid drug stored within the device to the patient's body. In this embodiment, the feedthrough can take the form of a cylindrical ceramic monolith, with the planar surfaces being the first and second surfaces. While the first and second surfaces are pure HTCC and LTCC respectively, HTCC and LTCC are interspersed between the two surfaces, with the area equidistant between the first and second surfaces being roughly half LTCC and half HTCC. The passageway connection exists between the first and second surfaces, with the pump included in the connection in a pure LTCC portion of the monolith proximate to the second surface. In one embodiment, the feedthrough is intended to contain no chemicals in excess of the limits imposed by the European Union Restriction of Hazardous Substances Directive.

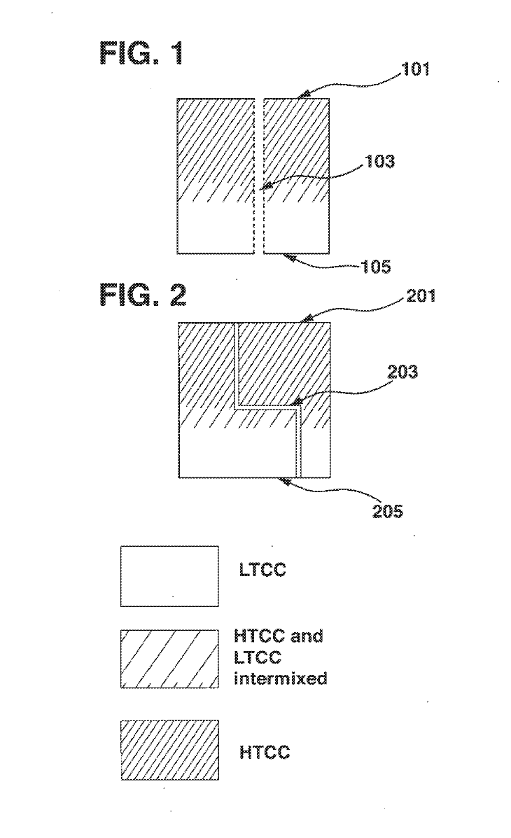

In one embodiment, the first and second surfaces of the ceramic monolith are substantially parallel, such that the feedthrough operates across opposite sides of the monolith through the center such as, for example, those shown in FIG. 1 or FIG. 2. FIG. 1 is a cross-section of one embodiment of a ceramic monolith of the present invention, viewed in cross section. FIG. 1 includes the first surface 101, the second surface 105, and the linear passageway 103 between the first and second surfaces. FIG. 2 is a cross-section of another embodiment of a ceramic monolith of the present invention with similar features to that of FIG. 1, except that the passageway 203 follows a non-linear path. FIG. 2 includes the first surface 201, the second surface 205, and the passageway between the first and second surfaces 203.

In one embodiment, the two ends of the passageway are both on the same surface of the monolith, as shown in FIG. 4. FIG. 4 is a cross-section of one embodiment of the present invention. FIG. 4 includes a substantially HTCC portion 401, a substantially LTCC portion 405, and the passageway between the HTCC and LTCC portions 403 that has both openings on the same surface of the monolith.

In one embodiment, the area of approximately equal LTCC-HTCC intermixing is not a plane, and may even extend along a passageway through the feedthrough.

In other embodiments, the first and second surfaces of the ceramic monolith are substantially perpendicular or otherwise not parallel, for example, as shown in FIG. 5. FIG. 5 is a cross-section of one embodiment of the present invention. FIG. 5 includes the first surface 501, the second surface 505, and the passageway between the first and second surfaces 503.

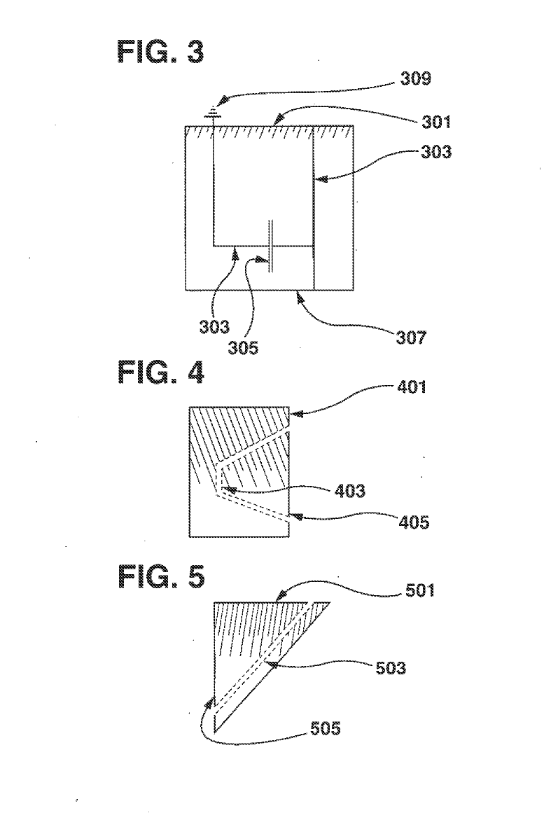

In one embodiment, the feedthrough is an electrical connection or set of connections between the first and second surfaces, for example, as shown in FIG. 3. These electrical connections may include components such as but not limited to capacitors, resistors, fuses, transistors, integrated circuits, and diodes. This electrical connection may even change the exact form of electrical transmission: a frequency filter may be included, a multiplexer, or an analog-digital converter. The electrical connection may be adapted to transmit energy, and the monolith may have the ability to power its own circuitry from the electrical connection or another source of energy, and may provide a ground connection. The electrical connection may follow a non-linear path, and include components which break a continuously conductive connection, such as capacitors or LED/photoresistor pairs.

FIG. 3 is a cross-section of one embodiment of the present invention including a low-pass filter. FIG. 3 includes the first surface 301, the second surface 307, the electrical interconnection between the first and second surfaces 303, a capacitor in electrical communication with the electrical connection 305, and a ground 309.

The present invention includes a number of advantages over prior feedthroughs. The ceramic feedthroughs of the present invention can be thinner and smaller, with a reasonably attainable thickness of about 0.0381 cm compared to thicker prior feedthroughs. This improvement in size allows for smaller implantable devices, or for more efficient use of device space.

FIG. 7 is a flow chart detailing one embodiment of a method to produce a version of the present invention. In a first step, an unfired high temperature co-fired ceramic (HTCC) material is fired to form a HTCC portion having a first surface and a second surface 701 and creating a porous region 703 on the second surface of the HTCC portion. The porous region can be created simultaneously with the firing of the HTCC or can be created subsequent to the firing step. A passageway is then formed that extends from the first surface to the second surface of the HTCC portion 705. An unfired low temperature co-fired ceramic (LTCC) material is provided where the unfired LTCC material has a sintering temperature, a first surface, and a second surface 707. A passageway is formed in the unfired LTCC material that extends from the first surface of the unfired LTCC to the second surface of the unfired LTCC material 709. The LTCC can also incorporate components sensitive to temperatures in excess of the firing temperature in the LTCC. The unfired LTCC material and the HTCC portions are mated together such that the passageway region on the second surface of the HTCC portion aligns with the passageway region on the second surface of the unfired LTCC material to create a resulting unit with a passageway from the first surface of the HTCC portion to the first surface of the unfired LTCC 711. In one embodiment the passageways are electrical interconnects. In other embodiments, the passageways are throughbores. The resulting unit is then fired at a temperature higher than the sintering temperature of the unfired LTCC material such that the LTCC material infiltrates the second surface of the HTCC portion and creates an intermixed LTCC/HTCC portion, and the unfired LTCC material sinters into a LTCC portion 713.

It should be understood that the porous interface of the HTCC material can be prepared by preparing the HTCC structure pre-firing to result in a porous interface or by a post firing step, such as through abrasion of the fired HTCC material. The porous mating surface of the HTCC structure permits and promotes infiltration of a liquid-sinter glass phase which bridges the interface between the LTCC and HTCC structure. Possible post-firing steps to create the porous layer include but are not limited to abrasion, drilling, photo etching, laser ablation, light ablation, chemical action, plasma use, percussive force, and heat ablation.

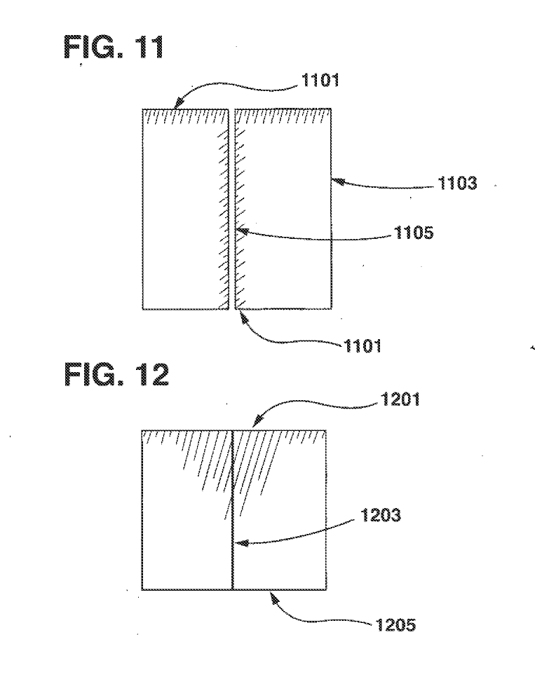

It should also be noted that depending on the geometry and function of the structure, the passageway and monolithic material may have different shapes, such as those shown in the various Figures. For example, FIG. 11 is one embodiment of the present invention with an HTCC lined aperture, including a first surface 1101 which extends along the exterior of the ceramic monolith and along an aperture 1105 running through the monolith from the first surface to the second surface, and a second surface 1103. FIG. 12 is one embodiment of the present invention with a non-planar LTCC-HTCC intermixing region, including a first surface 1201, a second surface 1205, and a passageway, such as a passageway embodied as an electrical connection 1203 extending from the first surface to the second surface.

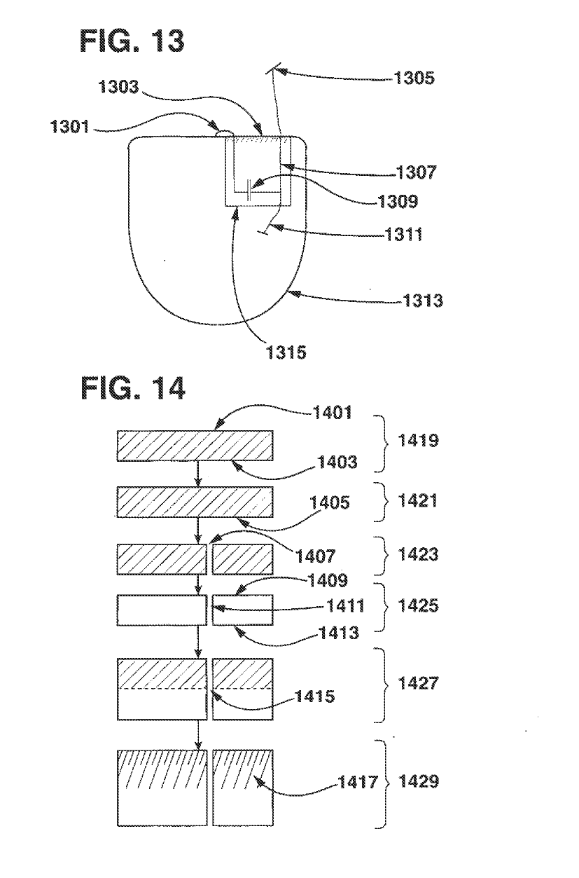

FIG. 13 shows an embodiment of a feedthrough of the present invention in a medical device. In FIG. 13, an implantable pacemaker device utilizes a feedthrough providing a low-pass filter. The device has a feedthrough with a first surface 1303, a second surface 1315, an electrical connection 1307, a capacitor 1309, a conductive lead to the heart 1305, a conductive lead to pacing circuitry 1311, a conductive device shell 1313, and a ground connection for the low-pass filter utilizing the conductive device shell 1301.

FIG. 14 is a visual flow chart illustrating steps in construction of one embodiment of the invention. In a first step 1419, a high temperature co-fired ceramic (HTCC) material is fired to form a HTCC portion having a first surface 1401 and a second surface 1403. In the next step 1421a porous region 1405 is created on the second surface. In the next step 1423, a passageway 1407 is formed extending from the first surface to the second surface of the HTCC portion. In the next step 1425 an unfired low temperature co-fired ceramic (LTCC) material is provided where the unfired LTCC has a sintering temperature, a first surface 1413, a second surface 1409, and passageway 1411 is formed. Electrical components can also be added to the LTCC material at this time. In the next step 1427 the unfired LTCC material and the HTCC portion are mated together such that the passageway region on the second surface of the HTCC portion aligns with the passageway region on the second surface of the unfired LTCC material to create a resulting unit with a passageway 1415. In the next step 1429, the resulting unit is fired at a temperature higher than the sintering temperature of the unfired LTCC such that the LTCC material infiltrates the second surface of the HTCC portion and creates a blended interface 1417.

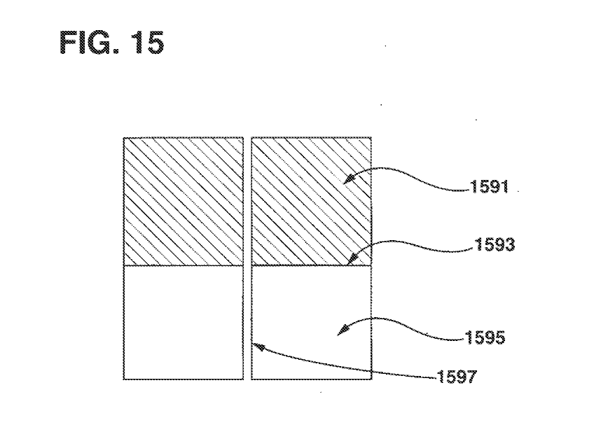

FIG. 15 is a cross-section of one embodiment of a prior art ceramic feedthrough. A substantially HTCC portion 1591 and a substantially LTCC portion 1595 are attached using an adhesive layer 1593 penetrated such that a passageway 1597 penetrates both regions.

The described method reduces the number of processing steps involved in producing a feedthrough. Electrical components do not have to be installed after firing of the monolith as in many prior feedthroughs, and the fired LTCC material does not have to be affixed or laminated to the HTCC after the LTCC is fired. Firing the LTCC with the already-fired HTCC can prevent some problems with shrinking evident in many feedthroughs.

The firing of the LTCC while already mated to the HTCC and with electrical components integrated allows reliable mating of electrical contacts and allows closer spacing of electrical connections.

The process described can be automated more easily than prior methods for creating other feedthroughs.

The claimed device provides advantages in forming a hermetic seal over the prior art. In prior feedthroughs, electronic components such as capacitors are often incorporated into the pin assembly in a way that can mask a defective hermetic seal. The ability to include these devices pre-firing eliminates this. More particularly, a defective braze or a defective glass-based seal structure, which would permit undesirable leakage of patient body fluids when mounted on a medical device and implanted into a patient, can be obstructed by the mounting of the filter capacitor and its associated electromechanical connections. For example, with reference to the feedthrough filter capacitor shown in U.S. Pat. No. 4,424,551, a ceramic filter capacitor is bonded to a glass seal and then embedded in epoxy material. Typical post-manufacture leak testing is performed by mounting the feedthrough assembly in a test fixture, and then subjecting one side of the feedthrough assembly to a selected pressurized gas such as helium. While the bulk permeability of the epoxy material is such that helium under pressure can penetrate therethrough in the presence of a defective hermetic seal, the duration of this pressure test (typically a few seconds) is often insufficient to permit such penetration. Accordingly, the epoxy material can mask the defective hermetic seal. The claimed device would allow any electronic components to be positioned within the monolith away from the pin assembly, allowing simplified and enhanced hermeticity testing.

In one embodiment, the passageway is an aperture used as an ink conduit in a printer or as a nozzle in an atomizer.

In one embodiment, the monolith is adapted to serve as an electrical feedthrough for an implantable medical device such as a pacemaker, defibrillator, or neurostimulator. This embodiment would likely include a capacitor to filter electromagnetic interference, or possibly more complex circuitry such as a band-pass filter to allow use of an antenna outside the main case of the implanted device.

In one embodiment the passageway is a connection intended to pass light, such as a fiber optic cable or window. Such a passage would have uses including but not limited to utilizing a camera, utilizing a light sensor, and utilizing a light emitter.

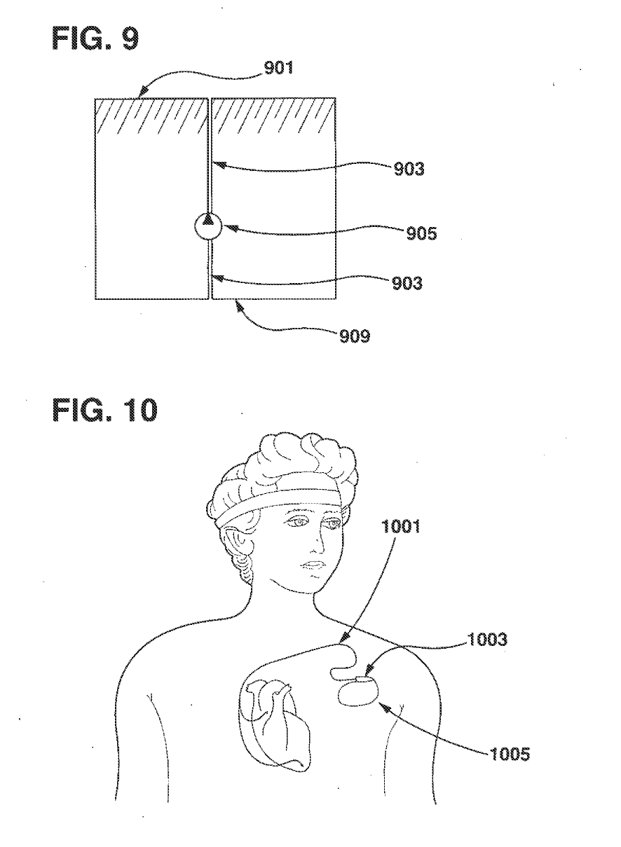

In one embodiment, the present invention is an implantable medical device for chemical delivery, utilizing the feedthrough as a port for transmission of one or more chemicals. In this embodiment, the feedthrough is an aperture extending from the inside of the implantable medical device to the patient's body, and the aperture serves as a route for chemicals to be expelled from the implantable medical device into the body or for chemicals to travel from the patient's body into the implantable medical device. The aperture may be lined with a number of substances, including HTCC, and there may be some types of hatch, valve, pump, or other device included in the passageway or on the exterior surface of the device. FIG. 9 is one embodiment of the present invention viewed in cross section. The embodiment illustrates a feedthrough for an implantable medical device for chemical delivery, including a first surface to contact the patient's body 901, a passageway for chemical movement 903, a pump 905, and a second surface to contact the interior of the device 909. The passageway may contain a gel, membrane, porous material, or other filling, which may be semi-permeable, and could be adapted to serve as a feedthrough that selectively allows venting of gas or liquid produced within the device. For example, the filling could be adapted to allow hydrogen gas, a byproduct of electrolyte breakdown in capacitors to escape, yet not allow electrolyte to leak through. Electrical bias may be used to move chemicals through the passageway. Magnetic bias may also be used to move chemical through the aperture.

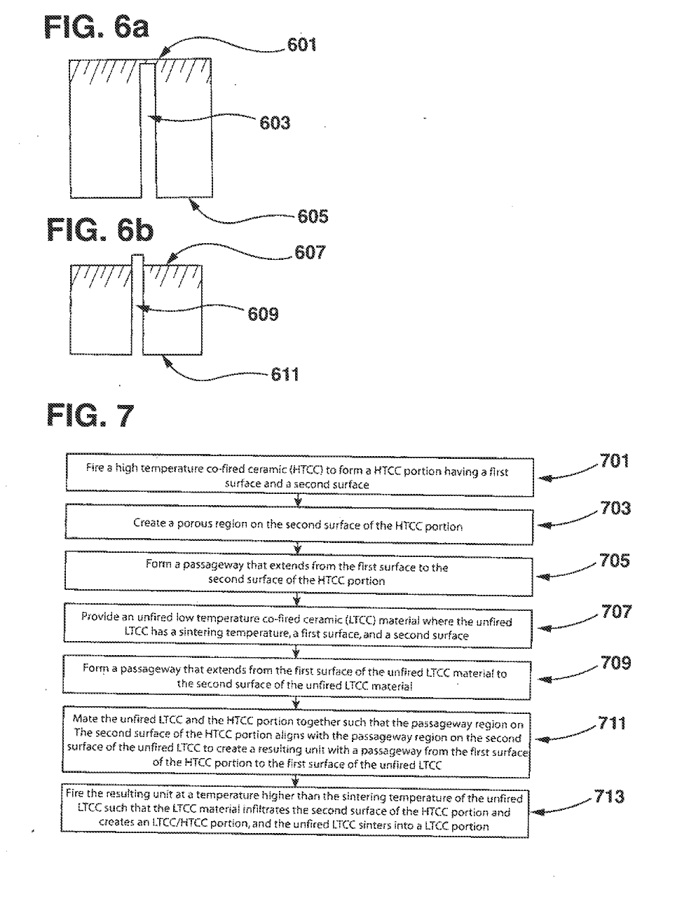

FIGS. 6a and 6b are cross-sections of embodiments of a feedthrough of the present invention. The embodiments include a passageway 603 that does not extend completely from second to first surface, a metal passageway 609 sealed on one end and extending from the second surface 611 to beyond the first surface 607, a first surface 601, and a second surface 605. The embodiment could be used to place a sensor close to a patient's body while maintaining a hermetic seal.

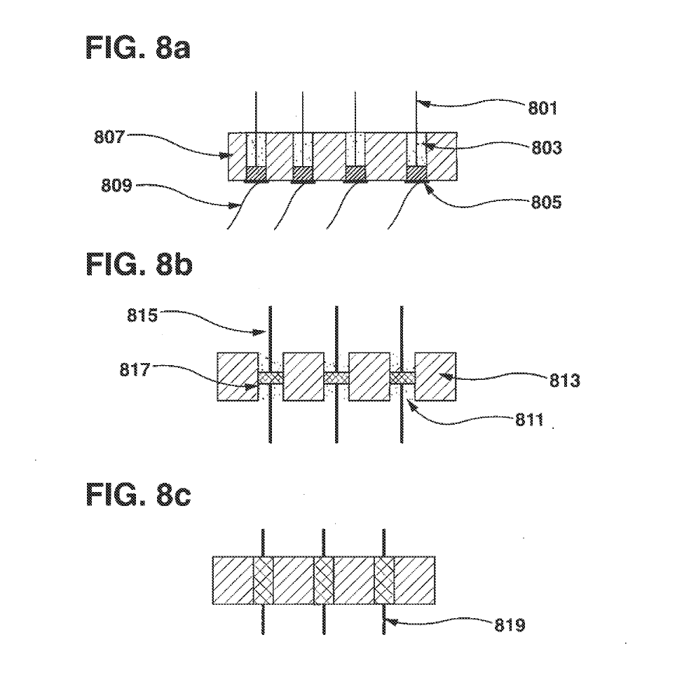

FIGS. 8a, 8b, and 8c are cross sectional views of three embodiments of electrical interconnects for HTCC portions of the LTCC-HTCC monolith, including a niobium, tantalum, or platinum pin 801, a bio-compatible solder 803, a platinum filled via 805, a HTCC substrate 807, a solder 811, a HTCC substrate 813, a niobium, tantalum, or platinum pin 815, a platinum filled via 817, and a pin attached to a platinum pad through percussion arc welding 819.

In one embodiment, the present invention is a medical device including a feedthrough as described herein. For example, FIG. 10 illustrates an embodiment of an implanted cardiac pacemaker unit. FIG. 10 depicts an implanted cardiac pacemaker unit comprising a lead connecting the HTCC surface of the feedthrough to the body 1001, a hybrid ceramic feedthrough 1003, and cardiac pacemaker electronic body 1005.

In one embodiment, the present invention is an implantable medical device including a feedthrough with an aperture adapted to vent any gases created inside the implantable medical device. This gas may be but is not limited to hydrogen created through operation of a battery in the implantable medical device.

In one embodiment, one or more capacitors included in the monolith use the monolith's LTCC or HTCC as a dielectric.

As used herein, the word “monolith” is defined as an object comprising a single, joined body. This body does not have to be homogenous, may be connected to other objects, and may have external features or a non uniform surface.

As used herein, the word “passageway” is defined as some type of connection, communication, or throughbore between two points which allows transmissions to pass, be they electrical, physical, optical, chemical, or other.

As used herein, the word “aperture” is defined as a physical connection between two points capable of transmitting liquids, solids, or gases. Any known ceramic materials can be utilized in the present invention.

A non-exhaustive list of HTCC materials includes: Hydroxyapatite, Boron nitride, Silicon Aluminium Oxynitrides, Silicon carbide, Silicon nitride, Zinc Oxide, Zirconia, Partially stabilized Zirconia, Al₂O₃, and Y₂O₃

A non-exhaustive list of LTCC materials includes: Lead zirconate titanate, Barium Titanate, Bismuth strontium calcium copper oxide, Ferrite, MgB₂, Titanium carbide, Yttrium barium copper oxide, Al₂O₃, and Zirconia-Toughened Alumina.

Depending on area of use, HTCC materials may be used as the LTCC component of the present invention, or LTCC materials may be used as the HTCC component of the present invention. The HTCC/LTCC distinction made in the paragraphs above is merely a reflection of sintering temperatures, and is not a bar for usage.

The explanations and illustrations presented herein are intended to acquaint others skilled in the art with the invention, its principles, and its practical application. Those skilled in the art may adapt and apply the invention in its numerous forms, as may be best suited to the requirements of a particular use. Accordingly, the specific embodiments of the present invention as set forth are not intended as being exhaustive or limiting of the invention.

I claim:

1. A ceramic monolith, comprising:
a) a first surface comprising a high temperature co-fired ceramic (HTCC);
b) a second surface comprising a low temperature co-fired ceramic (LTCC);
c) a passageway extending from the first surface to the second surface; and
d) a blended interface located between the first and second surfaces, the interface comprising intermixed HTCC and LTCC, wherein there is more LTCC disposed proximate to the second surface than proximate to the first and more HTCC disposed proximate to the first surface than proximate to the second surface.

2. The ceramic monolith of claim 1, wherein the LTCC and HTCC are interspersed within the monolith such that the ratio of LTCC to HTCC increases with distance from the first surface toward the second surface.

3. The ceramic monolith of claim 1, wherein the first and second surfaces are substantially parallel to one another

4. The ceramic monolith of claim 1, wherein the first and second surfaces are substantially perpendicular to one another

5. The ceramic monolith of claim 1, wherein the passageway does not extend out of the first surface.

6. The ceramic monolith of claim 1, the monolith further comprising a ceramic or metal augmenting member within the monolith.

7. An implantable medical device comprising a feedthrough, the feedthrough comprising:
a) a first surface comprising high temperature co-fired ceramic (HTCC);
b) a second surface comprising low temperature co-fired ceramic (LTCC); and
c) a blended interface located between the first and second surfaces, the interface comprising intermixed HTCC and LTCC such that there is more LTCC disposed proximate to the second surface than proximate to the first surface and more HTCC disposed proximate to the first surface than proximate to the second surface.

8. The implantable medical device of claim 7, wherein the feedthrough is an electrical feedthrough and comprises an electrical connection extending between the first surface and the second surface.

9. The implantable medical device of claim 8, further comprising one or more electrical components within the electrical feedthrough and in communication with the electrical connection.

10. The implantable medical device of claim 8, wherein the electrical feedthrough includes at least one capacitor connected to an electrical ground as a part of the electrical connection.

11. The implantable medical device of claim 8, wherein the electrical connection is adapted to serve as a conduit for transmission of electrical energy.

12. The implantable medical device of claim 8, further comprising a pattern of electrical connections in a plane between the first and second surfaces.

13. The implantable medical device of claim 7, wherein the feedthrough is a chemical feedthrough and comprises a passageway extending between the first surface and the second surface.

14. The implantable medical device of claim 13, wherein the passageway is an aperture extending from the first surface to the second surface.

15. A method of making a ceramic monolith, the method comprising:
g) firing a high temperature co-fired ceramic (HTCC) material to form a HTCC portion having a first surface and a second surface, the second surface having a porous region;
h) forming a passageway that extends from the first surface to the second surface of the HTCC portion;
i) providing an unfired low temperature co-fired ceramic (LTCC) material where the unfired LTCC has a sintering temperature, a first surface, and a second surface;
j) forming a passageway that extends from the first surface of the unfired LTCC material to the second surface of the unfired LTCC material;
k) mating the unfired LTCC and the fired HTCC portion together such that the passageway region on the second surface of the HTCC portion aligns with the passageway region on the second surface of the unfired LTCC to create a resulting unit with a passageway from the first surface of the HTCC portion to the first surface of the unfired LTCC; and
l) firing the resulting unit at a temperature higher than the sintering temperature of the unfired LTCC such that the LTCC material infiltrates the second surface of the HTCC portion and creates an LTCC/HTCC portion, and the unfired LTCC sinters into a LTCC portion.

16. The method of claim 15, the method further comprising introducing a conductor between the unfired LTCC material and the HTCC portion to aid formation of an electrically conductive connection between the first surface of the HTCC portion and the first surface of the unfired LTCC material.

17. The method of claim 15, wherein creating the porous region on the second surface of the HTCC portion is created by a method selected from the group consisting of: compressing the unfired HTCC material to create a density gradient before firing, using different sized granules throughout the HTCC before firing, including organic compounds in the HTCC material to burn out during firing, including unfired LTCC material in the HTCC material to burn out during firing, abrading the second surface of the HTCC portion after firing, including inorganic material in the unfired HTCC to burn out during firing, plasma treating the second surface of the HTCC portion after firing, chemical etching the second surface of the HTCC portion after firing, optically ablating the second surface of the HTCC portion after firing, heat ablating the second surface of the HTCC portion after firing, and combinations thereof.

18. The method of claim 16, further comprising mating the unfired LTCC and the HTCC portion together such that the electrically conductive region on the second surface of the HTCC portion aligns with the electrically conductive region on the second surface of the unfired LTCC to create a resulting unit with an electrically conductive connection from the first surface of the HTCC portion to the first surface of the unfired LTCC.

19. The method of claim 15, further comprising mating the unfired LTCC and the HTCC portion together such that the passageway on the second surface of the HTCC portion aligns with the passageway on the second surface of the unfired LTCC to create a resulting unit with a passageway from the first surface of the HTCC portion to the first surface of the unfired LTCC.

20. The method of claim 15, wherein the step of forming a passageway that extends from the first surface to the second surface of the HTCC portion occurs before firing the HTCC material.
